(19) United States
(12) Patent Application Publication
(10) Pub. No.: US 2010/0016438 A1
(43) Pub. Date: Jan. 21, 2010

(54) Granulated crystal compilation composed of pure organic minerals used in all food applications resulting in energy supplementation, diet and weight regulation, and medicinal effects

(76) Inventor: Daniel Hassani, Lake Elsinore, CA (US)

Correspondence Address: Daniel W. Hassani, 18495 Crestline Dr., Lake Elsinore, CA, 92532, US

(21) Appl. No.: 12/153,790

(22) Filed: Jul. 19, 2008

(51) Int. Cl. A61K 31/194; A23L 1/304

(52) U.S. Cl. 514/574; 426/2; 426/74

(57) ABSTRACT

A composition made of pure natural minerals that can be used in all food applications resulting in energy supplementation, diet and weight regulation, and medicinal benefits.

### CROSS-REFERENCE TO APPLICATION

N/A

### STATEMENT REGARDING FEDERALLY SPONSORED RESEARCH OR DEVELOPMENT

N/A

### REFERENCE TO A MICROFICHE APPENDIX

N/A

### BACKGROUND OF THE INVENTION

This invention relates to compositions of various minerals that produce advantageous beneficial effects in the form of energy derivation, proper nutrition cognizance, and energy supplementation.

The human body has a natural process in which it creates, utilizes, recycles, and reintegrates energies in the forms of complex molecules (in this aspect we are looking at the specific dormant energy containment structures known as complex carbohydrates or complex sugars (also known as fats/lipids). As one consumes food the body's digestive system breaks it down into later used for energy derivation.

The citric acid cycle is a reoccurring cycle in the human body which takes complex molecules and manipulates them into usable energies. The leftovers from the energy producing reaction are then recycled back into reusable energy molecules ready for the reaction again. This reaction supplied a constant supply of energy for the body to use as long as there is enough of the specific compounds (citric acid/citrates) present in the body heeded to initiate the reaction cycle process.

Citric Acid, as well as all citrates, posses the similar molecular structure and composition as the molecule citrate. Citrate is a specific initiating molecule present in the citric acid cycle process. In fact, citrate is the process initiating molecule resulting in a constant supply of energy as long as citrate is constantly being reintroduced into the cycle.

Citric Compound* contains the active ingredient/s such as Component A, described in detail on page 15 in the detailed description section, and when consumed and digested will release citric acid and citrates into the body for beneficial effects explained through out this application and in detail in the Summary of the Invention section.

### SUMMARY OF THE INVENTION

It is the objective of the proposed invention (Citric Compound*) to provide a variety of products to provide a healthy, all natural solution to modern day problems in the areas of energy, diet, medicine.

### Blood and Circulatory Benefits Produced by Consumption of Citric Compound*

When the Citric Compound* is consumed it like all consumables makes its way eventually to the blood stream by absorption into the blood at the small intestine. Once in the blood stream the Citric Compound* begins strengthening, filtering, and optimizing of the complete circulatory system. Like in other places of the body Citric Compound* upon contact of lipids, adipose tissues, fats, and/or cholesterol begins deteriorating them, breaking them up into more easily filterable material. This includes such complex lipids as cholesterol which by its natures collects in the bloodstream increasing blood pressure and contribution to numerous circulatory and cardiac ailments. The consumption and introduction of Citric Compound* into the bloodstream can significantly reduce the amount of these lipids in the blood vessels in turn lowering blood pressure and decreasing the risk percentage of circulatory and cardiac ailments. The presence of Citric Compound* in the blood also kills and reduces the number of impurities such as bacteria, minor virus' and Other Contaminants*** .

### Diet Advantages and Weight Loss Contributed to the Consumption of Citric Compound*.

While the circulation of the Citric Compound* in the body's destroys cholesterol in the blood vessels it will eventually be present throughout the entire bloodstream. After some time the Citric Compound* will be distributed into areas of lipid/adipose tissue concentration and there will exhaust its remaining strength to dissolve adipose tissues post and prior to adipose solidification in these concentrated areas. This in turn results in the decrease of the overall body fat percentage producing noticeable weight loss. As long as the Citric Compound* is in circulation in the blood and comes in contact with adipose complexes it will dissolve/breakdown them until the concentration of Citric Compound* in the blood decreases to the point where the neutralization reaction with the adipose tissues concludes. This conclusion point varies from person to person. By the nature of the Citric Compound* it dissolves fats/ and oils and all other types of lipids on contact. If Citric Compound* is added to foods prior to consumption this reaction will commence as well as in the body. This will eventually render the foods that had Citric Compound* added to them actually contain less lipid amounts then just previously before the Citric Compound* was added. This can lead to foods being prepared normally but simply by adding certain amounts of Citric Compound* to the preparation of the food will result in the completed food containing less lipid quantity than if Citric Compound* was not added in the preparation.

### Energy Supplementation Contributed to Consumption of Citric Compound*

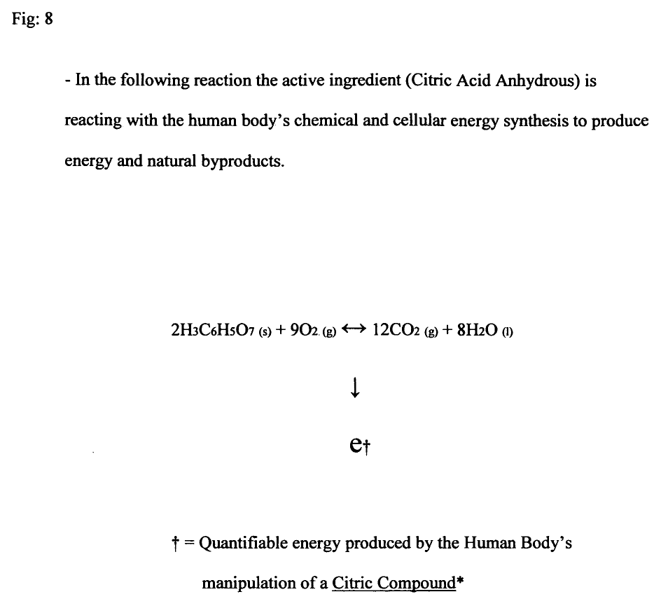

Depending on the type of food one consumed the percentage of energy derived from consumption varies. Some foods can be difficult to digest than the body may spend a lot of energy for digestion. In fact in many cases people spend more energy on digesting a certain food substance than they would gain from it. The consumption of Citric Compound* will result in a high gain of energy due to it being extremely easy to digest and its release of citrate into the blood. By itself the Citric Compound* can be used as a source of raw energy independent of a food substance. The Citric Compound* also aids in the digestive process by being present in the stomach with food substances it will assist in the breakdown of foods in addition to the stomachs on process'. The digestion of foods taken with Citric Compound* will require much less time and energy to digest resulting in a surplus of energy ordinarily needed for digestion. Since the Citric Compound* present with the food substance supplies energy of its own on top of the energy derived from ingestion of the food less food is required to derive required energy. The body will react physiologically by decreasing the appetite of the person since enough energy has been obtained by a lesser quantity of food. By consuming Citric Compound* with food more energy will be derived from smaller portions of food. As appetite decreases less food will be desired furthermore increasing weight loss and if desired one can use the appetite decreasing effects to aid in diet regimens. In addition, when the Citric Compound* is consumed energy can also be received on a microcellular level through cellular synthesis (FIG. 8 on page 9), resulting in an overall loss of lipids/adipose tissues throughout the entire body ultimately lowering the body fat percentage.

### Medicinal Advantages Contributed to the Consumption of Citric Compound*.

Since the Citric Compound* can easily get into the bloodstream and filter out impurities and lipid build up a better flow of blood in the entire circulatory system is to be expected. This alone has excellent medicinal properties. More oxygen that is distributed throughout the body will increase lipid/adipose synthesis into energy, stimulate muscle growth, decrease arthritis symptoms, and even aid in proper blood sugar regulation.
Citric Compound*—pertains to any combination, variation, and/or quantity of the chemical molecules specified in the Component A category.
Other Contaminants***—pertains to any contaminants in the bloodstream that can be rendered inactive due to pH neutralization when in contact with the Citric Compound*.

### DETAILED DESCRIPTION

The precise formula of the Citric Compound* can vary depending on desired affects but in order to be considered Citric Compound* the composition must contain Component A in the amounts specified in this detailed description. This is the root of the intended invention where all the objectives listed in the objectives section on page 19 are effects of consuming/using this Citric Compound* (minerals in the Component A Category).

### Component A:

The Component A category consists of minerals that makeup any percentage of the final composition from 1%-99%. The Component A category minerals are considered the Citric Compound* mentioned through this application. Component A/Citric Compound* is composed of the following minerals in any combination, arrangement, and/or variation, hydrated or not.

1. Citric Acid

2. Sodium Citrate.

3. MonoSodium Citrate

4. TriSodium Citrate

5. Potassium Citrate

6. MonoPotassium Citrate

7. TriPotassium Citrate

Parts B and C are buffing salts used to increase certain desired affects but is not essential to the Citric Compound* in any quantity.

### Component B:

The “Component B” category are minerals that make up any percentage of the final composition from 1%-80% and is composed of Potassium Chloride.

### Component C:

The “Component B” category are minerals that make up any percentage of the final composition from 1%-50% and is composed of Sodium Chloride.

### Optional Component D:

This category is made up of minor components such as vitamins and/or other minerals to supply additional effects that are desired. This category is not restricted to any percentages due to the variation of other minerals and vitamins.

Optimally a finished product claiming similar claims as the ones in this application must contain the mandatory Citric Compound*/Component A minerals. It may, depending on affects desired, contain a specific amount of both Category A and B minerals and also may or may not contain any Component D minerals/vitamins.

### DETAILED DESCRIPTION OF THE TABLES IN THIS APPLICATION

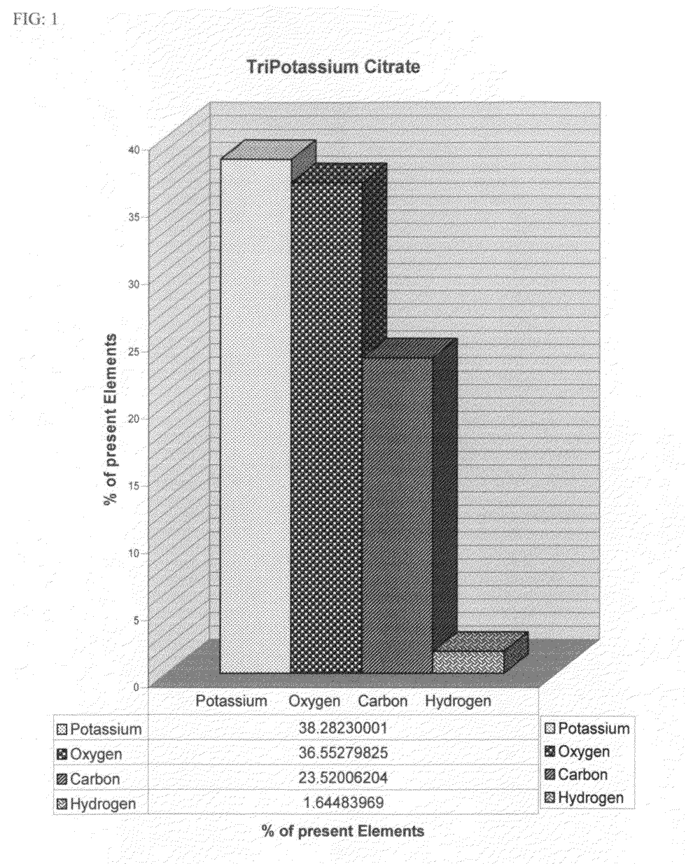

FIG. 1: Contains a detailed explanation of the percentages of specific elements in TriPotassium Citrate.

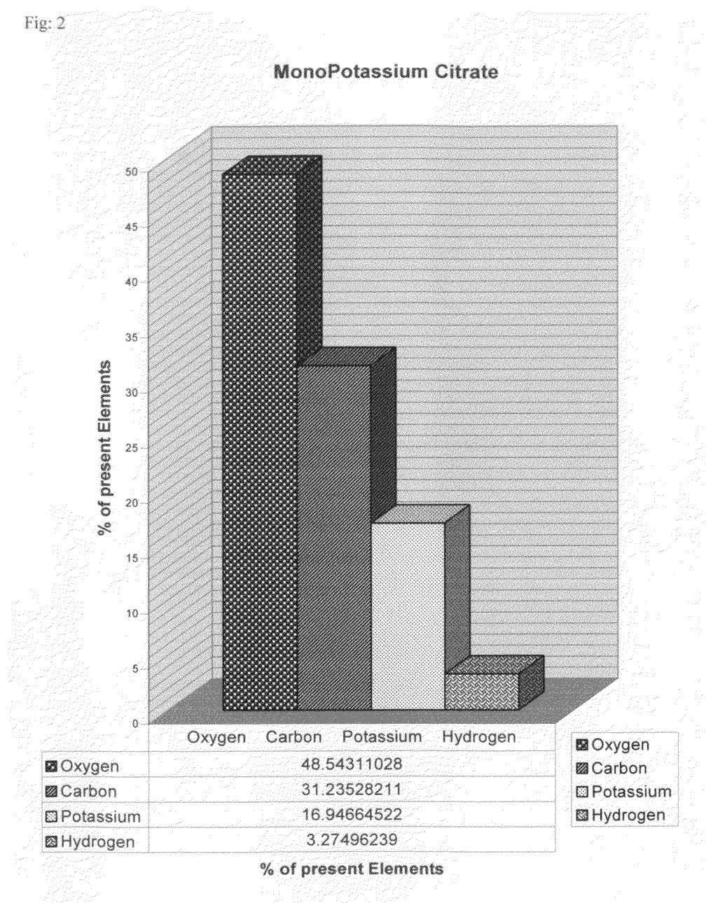

FIG. 2: Contains a detailed explanation of the percentages of specific elements in MonoPotassium Citrate.

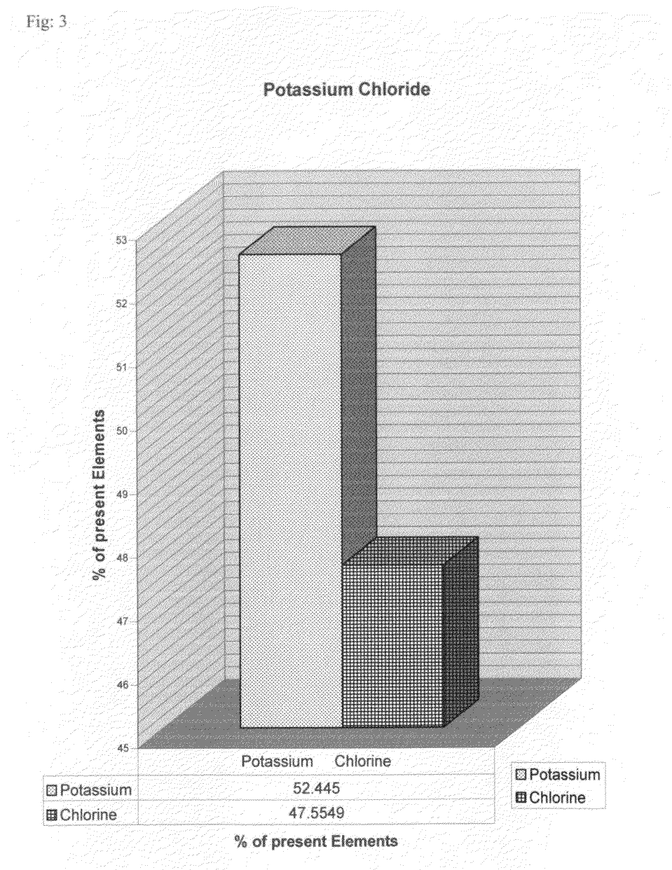

FIG. 3: Contains a detailed explanation of the percentages of specific elements in Potassium Chloride.

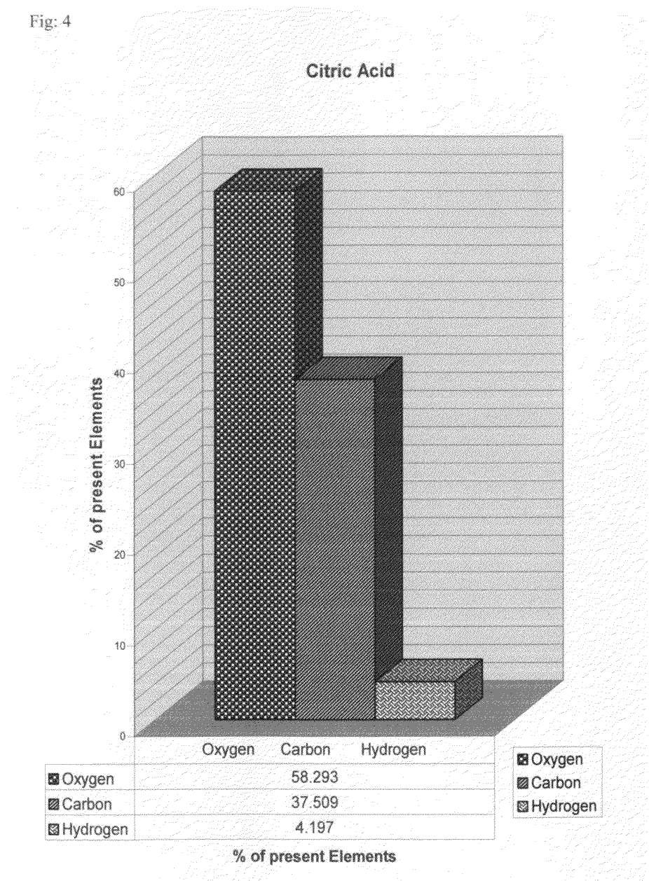

FIG. 4: Contains a detailed explanation of the percentages of specific elements in Citric Acid

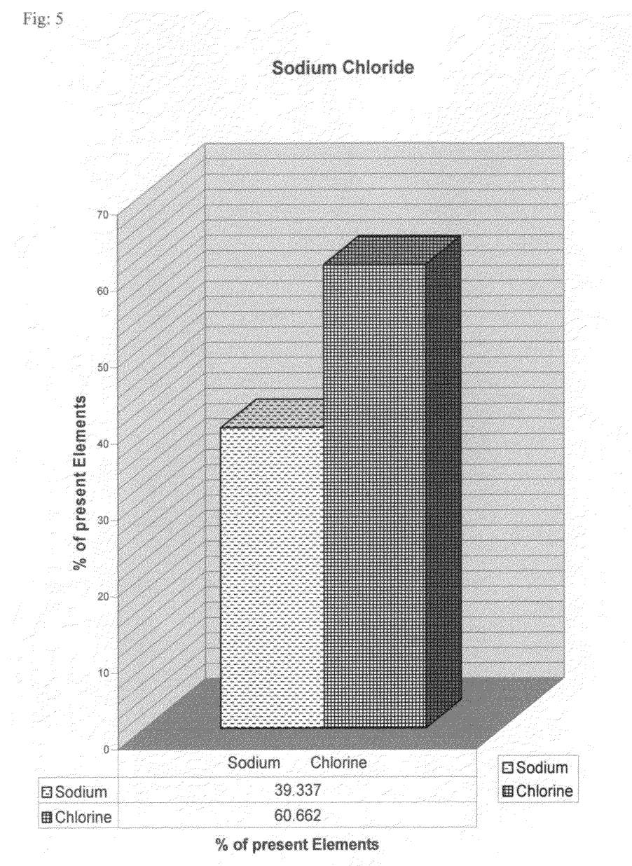

FIG. 5: Contains a detailed explanation of the percentages of specific elements in Sodium Chloride.

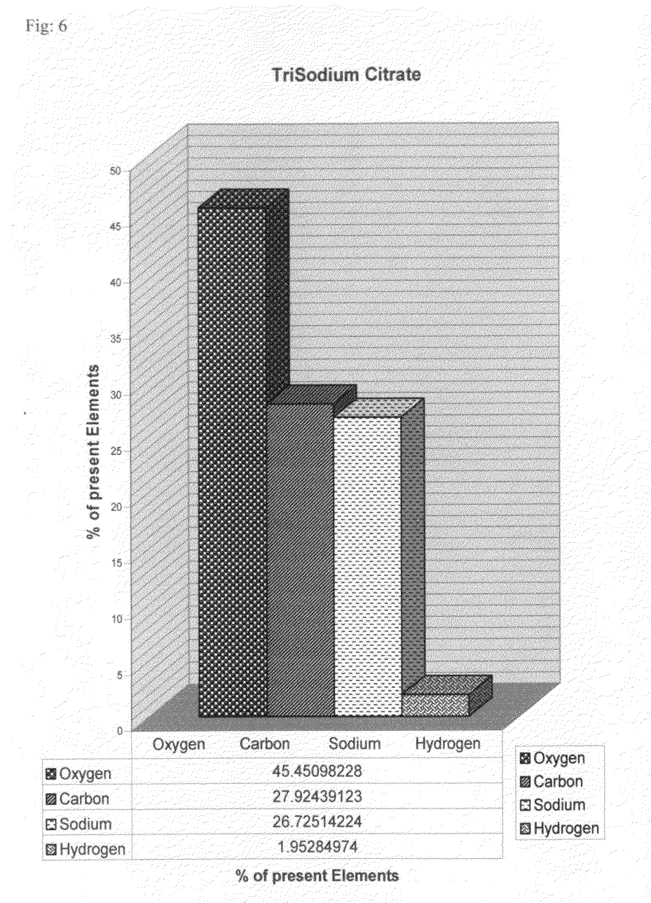

FIG. 6: Contains a detailed explanation of the percentages of specific elements in TriSodium Citrate.

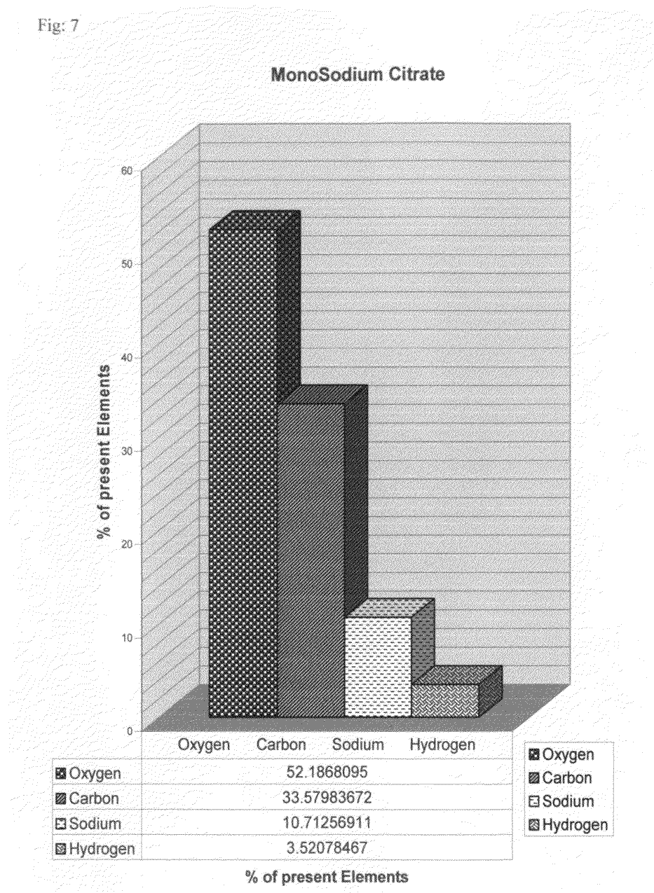

FIG. 7: Contains a detailed explanation of the percentages of specific elements in MonoSodium Citrate.

FIG. 8: Describes an example of energy synthesis through the breakdown of the molecule of Citric Acid.

The Citric compound* refers to the composition of selected minerals and complexes described in the detailed description starting on page 13 of the patent application. The selection, combination, and quantities of the minerals described vary depending on the type of application. The minerals described that make up the Citric compound* are usually produced in the forms of salts. The product form could vary however, for instance, if a liquid solution or gaseous solution were desired the Citric compound* could be produced in a gas and/or liquid form. These forms are not as wide spread as the standard crystalline form but will definitely play larger parts in future development of advanced products. The values given for the quantities of specific minerals in the detailed description starting on page 13 were derived using a standard 00 capsule dosage (1000 milligrams) which was encapsulated. This is one out of many ways the Citric compound* can be used. This method of use (encapsulated pill) does not restrict the Citric compound* to uses only by capsules, the Citric compound* can be consumed in any form (solid, liquid, and/or gas) and still achieve the objectives described below.

All of the claims listed below are affects that follow the introduction and/or consumption a Citric compound* serving. A serving size usually is between 500 to 1000 milligrams but depending on application the quantity can be different. In general though a 1000 milligram serving is taken orally, the serving can be encapsulated, liquefied, hydrated, made into tablets, or even made into table salt style dispensers.

1. By consuming* a composition consisting of one or more of the seven minerals listed on page 15 (Citric Acid, Sodium Citrate, MonoSodium Citrate, TriSodium Citrate, Potassium Citrate, MonoPotassium Citrate, and/or TriPotassium Citrate) in the amounts of no less than 2 mg and up to no more than 2000 mg per dosage* * per mineral the following affect will occur; the strengthening/optimizing of the physical and physiological condition and functionality of all cells(with the exception of fatty acids, cholesterol, and all other forms of lipids and oils present in the blood vessels)present throughout the circulatory system by aiding in the filtering out of impurities and free radicals additionally reconditioning the quality of red blood cells and plasma.

2. By consuming* a composition consisting of one or more of the seven minerals listed on page 15 in the amounts of no less than 2 mg and up to no more than 2000 mg per dosage** per mineral it will make its way into the blood stream through normal digestive means and once there dissolve fatty acids, cholesterol, and all other forms of lipids and oils present in the blood vessels.

3. A composition consisting of one or more of the seven minerals listed on page 15 in the amounts of no less than 2 mcg and up to no more than 2000 mg per dosage** per mineral will dissolve lipids, complex fats and complex carbohydrates upon physical contact with them and is not limited to reactions only in the human body, this can also happen externally on foods or product prior to consumption

4. A composition consisting of one or more of the seven minerals listed on page 15 in the amounts of no less than 2 mcg and up to no more than 2000 mg per dosage** per mineral will reduce quantifiable amounts of fat cells, complex/sugars, complex/carbohydrates, oils, and other lipids present in foods and products during and prior to consumption, digestion, and/or ingestion. Simply by its nature the composition deteriorates quantifiable amounts of fat cells, complex/sugars, complex/carbohydrates, oils, and other lipids.

5. A composition consisting of one or more of the seven minerals listed on page 15 in the amounts of no less than 2 mcg and up to no more than 2000 mg per dosage** per mineral will breakdown fat cells, lipids, oils, and/or complex sugars/carbohydrates present throughout the digestive system emphasizing the mouth, esophagus, stomach, small/large intestine, and colon.

6. After consuming* a composition consisting of one or more of the seven minerals listed on page 15 in the amounts of no less than 2 mcg and up to no more than 2000 mg per dosage** per mineral one will experience a reduction in fat cells, complex/sugars, complex/carbohydrates, oils, and other lipids present in the body, since lipid, fat cell, complex/sugar, complex/carbohydrate, and oil deterioration throughout the body is eminent when consuming the composition.

7. When consumed* by itself a composition consisting of one or more of the seven minerals listed on page 15 in the amounts of no less than 2 mcg and up to no more than 2000 mg per dosage** per mineral will act as a source of potential energy. The human body can easily break this consumption into usable quantities of energy (see FIG. 8).

8. When consumed* a composition consisting of one or more of the seven minerals listed on page 15 in the amounts of no less than 2 mcg and up to no more than 2000 mg per dosage** per mineral will allow reduction of the size of the portion of food required to be “full” or the amount needed to reach the limit of desired consumption (the appetite quantity). By supplying energy of its own the composition energy charges the food substance present in the stomach. The body in turn responds physiologically by curbing the appetite since enough energy (food plus the composition) has been consumed. This consequently affects and lessens the serving portions of desired food quantities. This also results in less food required to derive the same amount of energy.

9. When consumed a composition consisting of one or more of the seven minerals listed on page 15 in the amounts of no less than 2 mcg and up to no more than 2000 mg per dosage** per mineral will conserve required energy needed in the process of digestion. Since the composition rapidly deteriorates all substances in the stomach present at the same time, the stomach actually spends much less time and energy to digests foods than normal. This in turn provides the body with a surplus of energy unused from the digestion process.

10. When consumed a composition consisting of one or more of the seven minerals listed on page 15 in the amounts of no less than 2 mcg and up to no more than 2000 mg per dosage** per mineral will breakdown food substances into more usable material. By pre-breaking down food substances the body is allowed to more completely digest more quantities of food substance than before. By aiding the body's digestion into taking full advantage of all the food substance not just a portion (normally) the affect is that more energy is derived from that food substance than would have been without the composition.
*consuming—the actual act of consumption varies for different consumer types and specific applications. Usually consumption is by means of a pharmaceutical grade encapsulation (pill) of various amounts. However, consumption vessels can also include tablets, salts, granules, powders, liquids, mixtures, solutions, crystalline, and gaseous in order to encompass a wider variety of applications.
** dosage—the actual dosage varies for different consumer types as well as specific application. Dosages can vary per specified regimens i.e. where a dietary supplement application will restrict dosage of one or more of the seven minerals listed on page 15 to the amounts of no less than 2 mg and up to no more than 2000 mg per dosage and limit the consumption interval to twice or three times daily. This can change considerably for instance as mentioned in claim 3 on page 19 the dosage of one or more of the seven minerals listed on page 15 can exceed the amounts of no more than 2000 mg per dosage due to being on an external application where considerable volumes of mineral will be neutralized prior to consumption.
